(19) United States
(12) Patent Application Publication
(10) Pub. No.: US 2017/0231470 A1
(43) Pub. Date: Aug. 17, 2017

(54) CAPSULE ENDOSCOPE SYSTEM, CAPSULE ENDOSCOPE, WIRELESS COMMUNICATION METHOD OF CAPSULE ENDOSCOPE, AND PROGRAM

(71) Applicant: OLYMPUS CORPORATION, Tokyo (JP)

(72) Inventor: Masaharu Yanagidate, Sagamihara-shi (JP)

(73) Assignee: OLYMPUS CORPORATION, Hachioji-shi, Tokyo (JP)

(21) Appl. No.: 15/584,385

(22) Filed: May 2, 2017

Related U.S. Application Data
(63) Continuation of application No. PCT/JP2014/080747, filed on Nov. 20, 2014.

(51) Int. Cl. A61B 1/00; A61B 1/04; A61B 1/045; A61B 5/06

(52) U.S. Cl. CPC A61B 1/00016; A61B 1/00158; A61B 5/065; A61B 5/067; A61B 1/041; A61B 1/045; A61B 1/00036; A61B 5/0013

(57) ABSTRACT

A capsule endoscope system includes a capsule endoscope and a receiving device. The capsule endoscope outputs execution command at a timing at which work instruction data is received when movement speed is low, and outputs the execution command at a timing at which output of the execution command is instructed when the movement speed is high. The receiving device generates the work execution condition data and the work instruction data based on operations of an operator, and transmits the work execution condition data and the work instruction data to the capsule endoscope.

The present application is a continuation application based on International Patent Application No. PCT/JP2014/080747 filed Nov. 20, 2014, the content of which is incorporated herein by reference.

### BACKGROUND OF THE INVENTION

Field of the Invention

The present invention relates to a capsule endoscope system, a capsule endoscope, a wireless communication method of the capsule endoscope, and a program.

Description of Related Art

A capsule endoscope system includes a capsule endoscope and a receiving device. The capsule endoscope includes an imaging unit that performs imaging and an acceleration sensor that detects acceleration. In addition, the capsule endoscope wirelessly transmits image data from the imaging unit and acceleration data from the acceleration sensor to the receiving device. The receiving device receives the image data and the acceleration data and stores the image data. In addition, the receiving device has a position detection function of detecting a position of the capsule endoscope in a human body. Moreover, the capsule endoscope has medication/examination functions, and executes medication/examination when the capsule endoscope reaches the vicinity of a lesion.

In Japanese Patent Application Publication No. 2005-185644, an example of a capsule medical device which includes a capsule endoscope transmitting information from an acceleration sensor disposed in a capsule endoscope to a receiving device outside a body, and includes a receiving device having a function of estimating a position of the capsule endoscope is disclosed.

### SUMMARY OF THE INVENTION

According to a first aspect of the present invention, a capsule endoscope system includes a capsule endoscope and a receiving device. The capsule endoscope includes an imaging unit which performs imaging and outputs image data, an acceleration sensor which outputs acceleration data, and a first wireless communication unit which transmits the image data and the acceleration data to the receiving device by wireless communication. The receiving device includes a second wireless communication unit which receives the image data and the acceleration data from the capsule endoscope by wireless communication, and a capsule position detection unit which detects a position of the capsule endoscope based on the image data and the acceleration data. The first wireless communication unit further receives work execution condition data and work instruction data from the receiving device, the work execution condition data represents a position at which treatment work is performed, and the work instruction data represents an execution instruction of the treatment work. The capsule endoscope further includes a speed/distance detection unit which detects a movement speed and a movement distance of the capsule endoscope based on the acceleration data, an execution timing decision unit which instructs output of an execution command at a timing based on the movement distance and the work execution condition data, a treatment work unit which performs medication or “collection of tissue or body fluid” based on the execution command, and a capsule control unit which outputs the execution command to the treatment work unit at a timing at which the work instruction data is received when the movement speed is low, and outputs the execution command to the treatment work unit at a timing at which output of the execution command is instructed by the execution timing decision unit when the movement speed is high. The receiving device further includes an operation unit which receives operations of an operator, and a generation unit which generates the work execution condition data and the work instruction data based on the operations received by the operation unit. The second wireless communication unit transmits the work execution condition data and the work instruction data generated by the generation unit to the capsule endoscope.

According to a second aspect of the present invention, in the first embodiment, the capsule endoscope further includes an acceleration data storage unit which temporarily stores the acceleration data, and a communication environment detection unit which detects a wireless communication environment. The capsule control unit causes the acceleration data storage unit to store the acceleration data when deterioration in the wireless communication environment is detected by the communication environment detection unit, and causes the first wireless communication unit to transmit the acceleration data stored in the acceleration data storage unit to the receiving device after recovery of the wireless communication environment is detected by the communication environment detection unit.

According to a third aspect of the present invention, in the second embodiment, the capsule control unit may output the execution command to the treatment work unit at the timing at which the work instruction data is received when the movement speed is low and deterioration in the wireless communication environment is not detected by the communication environment detection unit. The capsule control unit may output the execution command to the treatment work unit at the timing at which output of the execution command is instructed by the execution timing decision unit when the movement speed is low and deterioration in the wireless communication environment is detected by the communication environment detection unit.

According to a fourth aspect of the present invention, in the second or the third embodiment, the capsule endoscope may further include an image data storage unit which temporarily stores the image data output from the imaging unit. The imaging unit may also perform the imaging according to the movement distance from a position at which the execution command is output. The first wireless communication unit may also further transmit the image data stored in the image data storage unit to the receiving device.

According to a fifth aspect of the present invention, the capsule endoscope includes an imaging unit which performs imaging and outputs image data, an acceleration sensor which outputs acceleration data, a first wireless communication unit which transmits the image data and the acceleration data to the receiving device by wireless communication, and receives work execution condition data and work instruction data from the receiving device, the work execution condition data represents a position at which treatment work is performed, and the work instruction data represents an execution instruction of the treatment work, a speed/distance detection unit which detects a movement speed and a movement distance of the capsule endoscope based on the acceleration data, an execution timing decision unit which instructs output of an execution command at a timing based on the movement distance and the work execution condition data, a treatment work unit which performs medication or “collection of tissue or body fluid” based on the execution command, and a capsule control unit which outputs the execution command to the treatment work unit at a timing at which the work instruction data is received when the movement speed is low, and outputs the execution command to the treatment work unit at a timing at which output of the execution command is instructed by the execution timing decision unit when the movement speed is high.

According to a sixth aspect of the present invention, a wireless communication method of a capsule endoscope includes a step of performing imaging and outputting image data, a step of outputting acceleration data, a step of transmitting the image data and the acceleration data to the receiving device by wireless communication, and receiving work execution condition data and work instruction data from the receiving device, the work execution condition data represents a position at which treatment work is performed, and the work instruction data represents an execution instruction of the treatment work, a step of detecting a movement speed and a movement distance of the capsule endoscope based on the acceleration data, a step of outputting the execution command to the treatment work unit at a timing at which the work instruction data is received when the movement speed is low, and outputting the execution command to the treatment work unit at a timing at which output of the execution command is instructed by the execution timing decision unit when the movement speed is high, and a step of performing medication or “collection of tissue or body fluid” based on the execution command.

According to a seventh aspect of the present invention, a program is a program for causing a computer of a capsule endoscope to execute a step of performing imaging and outputting image data, a step of outputting acceleration data, a step of transmitting the image data and the acceleration data to the receiving device by wireless communication, and receiving work execution condition data and work instruction data from the receiving device, the work execution condition data represents a position at which treatment work is performed, and the work instruction data represents an execution instruction of the treatment work, a step of detecting a movement speed and a movement distance of the capsule endoscope based on the acceleration data, a step of outputting the execution command to the treatment work unit at a timing at which the work instruction data is received when the movement speed is low, and outputting the execution command to the treatment work unit at a timing at which output of the execution command is instructed by the execution timing decision unit when the movement speed is high, and a step of performing medication or “collection of tissue or body fluid” based on the execution command.

### BRIEF DESCRIPTION OF THE DRAWINGS

FIG. 1 is a block diagram which shows a configuration of a capsule endoscope system according to a first embodiment of the present invention.

FIG. 2 is a schematic diagram which shows a usage state of the capsule endoscope system according to the first embodiment of the present invention.

FIG. 3 is a block diagram which shows a configuration of a capsule endoscope according to the first embodiment of the present invention.

FIG. 4 is a block diagram which shows a configuration of a receiving device according to the first embodiment of the present invention.

FIG. 5 is a flowchart which shows a procedure of operations of the capsule endoscope according to the first embodiment of the present invention.

FIG. 6 is a block diagram which shows a configuration of a capsule endoscope system according to a second embodiment of the present invention.

FIG. 7 is a schematic diagram which shows a usage state of the capsule endoscope system according to the second embodiment of the present invention.

FIG. 8 is a block diagram which shows a configuration of a capsule endoscope according to the second embodiment of the present invention.

FIG. 9 is a block diagram which shows a configuration of a repeater according to the second embodiment of the present invention.

FIG. 10 is a block diagram which shows a configuration of an operation/saving device according to the second embodiment of the present invention.

FIG. 11 is a reference diagram which shows a state of the capsule endoscope when the capsule endoscope of the second embodiment of the present invention performs treatment work.

FIG. 12 is a reference diagram which shows an image captured during the treatment work by the capsule endoscope according to the second embodiment of the present invention.

FIG. 13 is a reference diagram which shows an image captured during the treatment work by the capsule endoscope according to the second embodiment of the present invention.

FIG. 14 is a flowchart which shows a procedure of operations of the capsule endoscope according to the second embodiment of the present invention.

FIG. 15 is a flowchart which shows a procedure of operations of the capsule endoscope according to the second embodiment of the present invention.

FIG. 16 is a block diagram which shows a configuration of a capsule endoscope according to a third embodiment of the present invention.

FIG. 17 is a flowchart which shows a procedure of operations of the capsule endoscope according to the third embodiment of the present invention.

### DETAILED DESCRIPTION OF THE INVENTION

Embodiments of the present invention will be described with reference to drawings.

### First Embodiment

A first embodiment of the present invention is an example in which the present invention is applied to a capsule endoscope system having a capsule endoscope and a receiving device. The capsule endoscope has an imaging unit which performs imaging and outputs image data, and an acceleration sensor which detects acceleration and outputs acceleration data. In addition, the capsule endoscope transmits the image data and the acceleration data to a receiving device by wireless communication. The receiving device receives the image data and the acceleration data from the capsule endoscope. Moreover, the receiving device has a function of calculating a position of the capsule endoscope in a human body using the received image data and acceleration data.

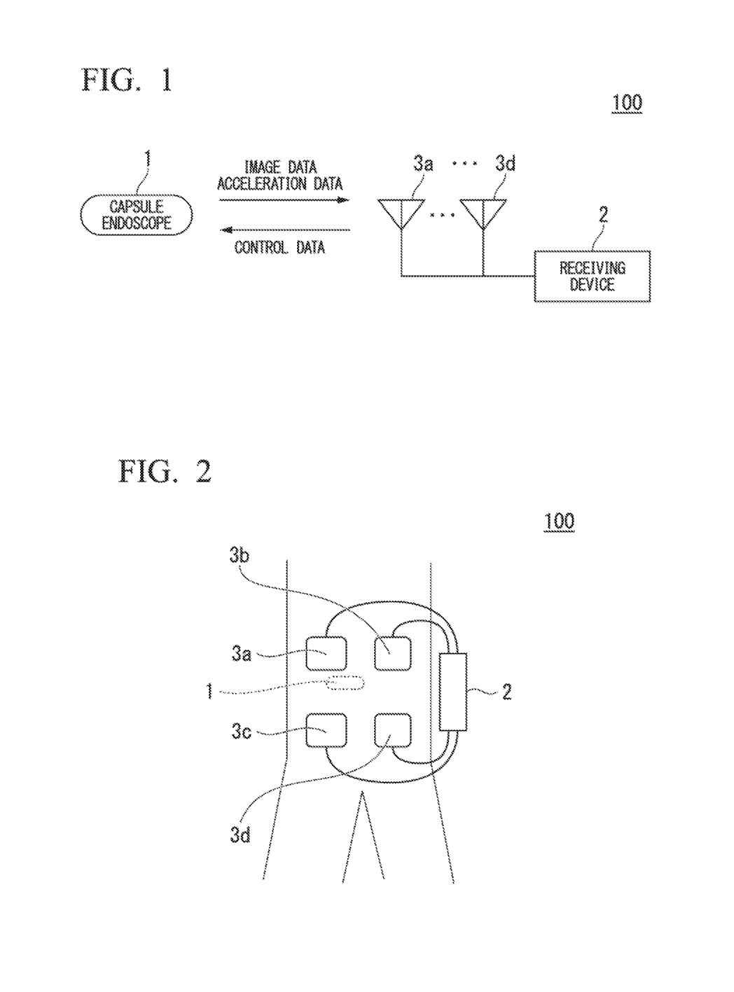

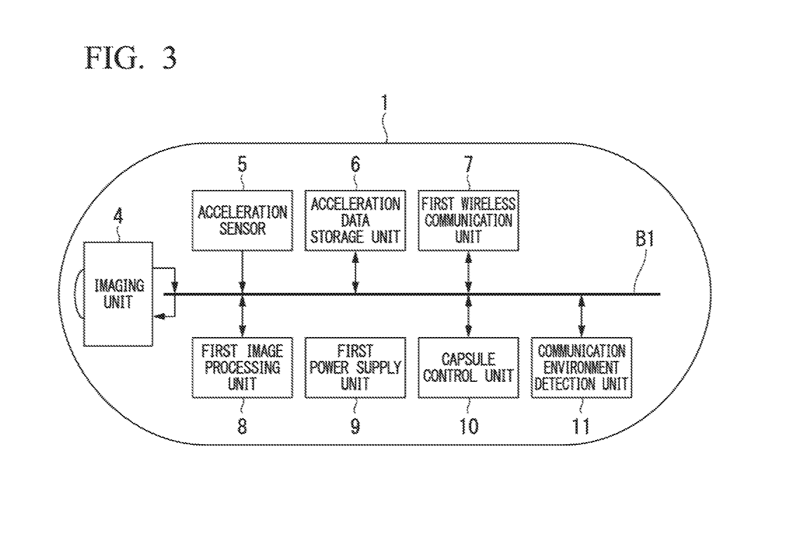

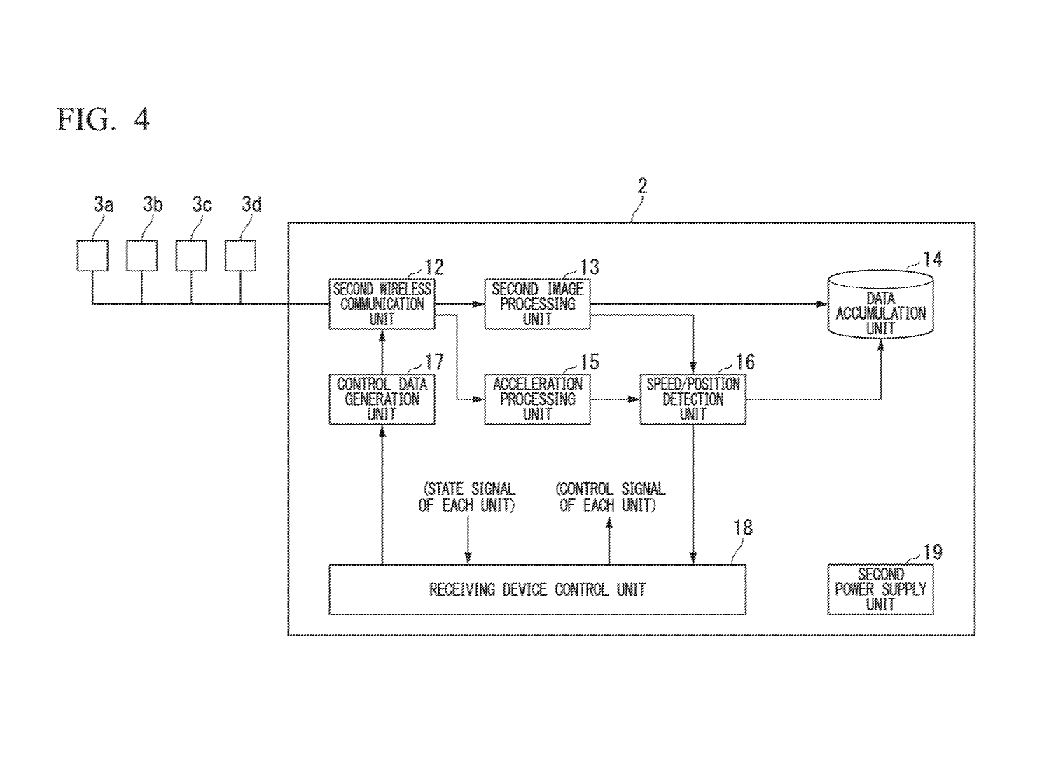

A system configuration and a device configuration will be described with reference to FIGS. 1 to 4. FIG. 1 shows a configuration of a capsule endoscope system 100. FIG. 2 shows a usage state of the capsule endoscope system 100. FIG. 3 shows a configuration of a capsule endoscope 1. FIG. 4 shows a configuration of a receiving device 2.

As shown in FIG. 1, the capsule endoscope system 100 includes the capsule endoscope 1 and the receiving device 2. The image data and the acceleration data are wirelessly transmitted from the capsule endoscope 1 to the receiving device 2. Control data for controlling a frame rate of an imaging unit included in the capsule endoscope 1 is wirelessly transmitted from the receiving device 2 to the capsule endoscope 1. Wireless communication between the capsule endoscope 1 and the receiving device 2 is performed via an antenna in the capsule endoscope 1 and antennas 3a to 3d of the receiving device 2. Only the antenna 3a and the antenna 3d are shown in FIG. 1.

FIG. 2 shows a state in which the antennas 3a to 3d are attached to a human body (patient) and shows a positional relationship between the capsule endoscope 1 and the receiving device 2. The capsule endoscope 1 operates for a long time with an internal battery. To this end, power used for the wireless communication is suppressed to the minimum. For this reason, the antennas 3a to 3d are used in a state in which they are attached to the human body so that a distance between the capsule endoscope 1 and the antennas 3a to 3d is the shortest distance.

As described above, the capsule endoscope 1 minimizes the power used for the wireless communication. Therefore, deterioration in a wireless communication environment occurs depending on a positional relationship between the capsule endoscope 1 and the antennas 3a to 3d attached to the human body and a state of the human body which is a communication path.

The receiving device 2 has a capsule position detection function of detecting a position of the capsule endoscope 1 in the human body from the received image data and acceleration data. Various methods of calculating a position of a capsule endoscope in the human body depending on the image data and the acceleration data have been devised. In embodiments of the present invention, a method of detecting a characteristic site such as a site at which organs meet (a junction) depending on the image data is adopted. Moreover, in this method, this site is a reference position, and the position of the capsule endoscope is detected by calculating an amount of movement from each reference position using the acceleration data.

When the acceleration data changes without any change in images in adjacent frames, this change is treated as a change in the acceleration data caused not by a movement of the capsule endoscope 1 but by a motion of the patient. This eliminates effects of patient motion and increases accuracy in position detection.

In the embodiments of the present invention, positional information obtained by the above method is stored associated with the image data. In addition, control data for controlling a frame rate of imaging according to a position of the capsule endoscope 1 is wirelessly transmitted.

As shown in FIG. 3, the capsule endoscope 1 includes an imaging unit 4, an acceleration sensor 5, an acceleration data storage unit 6, a first wireless communication unit 7, a first image processing unit 8, a first power supply unit 9, a capsule control unit 10, a communication environment detection unit 11, and a data bus B1.

The imaging unit 4 (imaging element) performs imaging and outputs image data. The imaging unit 4 performs imaging in a human body at a designated frame rate. The acceleration sensor 5 detects acceleration applied to the capsule endoscope 1 and outputs acceleration data. The acceleration sensor 5 periodically detects acceleration. The acceleration data storage unit 6 (storage medium) temporarily stores the acceleration data. The first wireless communication unit 7 (first wireless communication circuit) transmits the image data and the acceleration data to the receiving device 2 by wireless communication. In addition, the first wireless communication unit 7 receives the control data from the receiving device 2 by wireless communication. The first image processing unit 8 (first image processing circuit) performs image processing such as compression processing on the image data from the imaging unit 4. The first power supply unit 9 (first power supply circuit) supplies power to each unit.

The capsule control unit 10 (capsule control circuit) controls an operation of each unit. For example, when deterioration in a wireless communication environment is detected by the communication environment detection unit 11, the capsule control unit 10 causes the acceleration data storage unit 6 to store the acceleration data. In addition, the capsule control unit 10 causes the first wireless communication unit 7 to transmit the acceleration data stored in the acceleration data storage unit 6 to the receiving device 2 after recovery of the wireless communication environment is detected by the communication environment detection unit 11. In addition, the capsule control unit 10 detects a frame rate designation value from the received control data and sets a frame rate of the imaging unit 4 based on the frame rate designation value. The communication environment detection unit 11 detects the wireless communication environment from a communication state of the first wireless communication unit 7. The first image processing unit 8, the capsule control unit 10, and the communication environment detection unit 11 may also be configured with an integrated circuit such as a processor. The data bus B1 transfers various types of data.

The capsule control unit 10 stores a program and necessary data for controlling an operation of the capsule control unit 10. For example, a computer of the capsule endoscope 1 reads and executes a program including a command for specifying the operation of the capsule control unit 10, and thereby a function of the capsule control unit 10 may be realized as a software function. This program may also be provided on a “computer readable recording medium” such as a flash memory. In addition, the program described above may also be transferred to the capsule endoscope 1 from a computer having a storage device storing this program, and the like, through a transfer medium or by transfer waves in the transfer medium. The “transfer medium” which transfers the program is a medium having a function of transferring information, such as a network (communication network) such as the Internet or a communication channel (communication line) such as a telephone line. In addition, the program described above may realize some of the functions described above. Furthermore, the program described above may also be a so-called differential file (differential program) which can realize the functions described above in combination with a program previously recorded in the computer.

Since the capsule endoscope 1 operates in the human body, there is a limit on a size of the capsule endoscope 1. Accordingly, a capacity of a battery which is usable as the first power supply unit 9 is limited. Therefore, in shooting of an organ or a site which is not to be diagnosed, a frame rate is required to be lowered for power saving. Control data used to control the frame rate is transmitted from the receiving device 2 at a predetermined period.

For example, the communication environment detection unit 11 detects deterioration in the wireless communication environment from a reception status of the control data. A result of the detection by the communication environment detection unit 11 is represented as “good” or “deterioration.”

The capsule endoscope 1 is equipped with the acceleration sensor 5 which outputs acceleration data representing acceleration of the capsule endoscope 1. When a result of the detection by the communication environment detection unit 11 is “good,” time data representing detection timing is added to the acceleration data from the acceleration sensor 5. The acceleration data to which the time data is added is transmitted from the first wireless communication unit 7. When a result of the detection by the communication environment detection unit 11 is “deterioration,” the time data representing detection timing is added to the acceleration data. The acceleration data to which the time data is added is temporarily stored in the acceleration data storage unit 6. The acceleration data stored in the acceleration data storage unit 6 is transmitted from the first wireless communication unit 7 at a timing at which a result of the detection by the communication environment detection unit 11 is “good.” Therefore, the capsule control unit 10 causes the first wireless communication unit 7 to transmit the acceleration data stored in the acceleration data storage unit 6 to the receiving device 2 when recovery of the wireless communication environment is detected by the communication environment detection unit 11 after deterioration in the wireless communication environment is detected by the communication environment detection unit 11 and the acceleration data is stored in the acceleration data storage unit 6.

As shown in FIG. 4, the receiving device 2 includes the antennas 3a, 3b, 3c, and 3d, a second wireless communication unit 12, a second image processing unit 13, a data accumulation unit 14, an acceleration processing unit 15, a speed/position detection unit 16, a control data generation unit 17, a receiving device control unit 18, and a second power supply unit 19.

The antennas 3a, 3b, 3c, and 3d are wirelessly connected to the capsule endoscope 1. The second wireless communication unit 12 (second wireless communication circuit) receives the image data and the acceleration data from the capsule endoscope 1 by wireless communication. In addition, the second wireless communication unit 12 transmits control data to the capsule endoscope 1 by wireless communication. The second image processing unit 13 (second image processing circuit) performs image processing such as expansion processing on the image data received by the second wireless communication unit 12, and converts the image data into data in a format suitable for each unit. The image data processed by the second image processing unit 13 is output to the speed/position detection unit 16 and the data accumulation unit 14.

The acceleration processing unit 15 (acceleration processing circuit) converts the acceleration data received by the second wireless communication unit 12 into speed data and movement distance data at predetermined time intervals. The speed data and the movement distance data are output to the speed/position detection unit 16. The speed/position detection unit 16 (speed/position detection circuit) calculates position data and speed data based on the image data from the second image processing unit 13 and the speed data and the movement distance data at predetermined time intervals from the acceleration processing unit 15. The position data calculated by the speed/position detection unit 16 represents a position of the capsule endoscope 1 in the human body. The speed data calculated by the speed/position detection unit 16 represents a speed corresponding to the position of the capsule endoscope 1 in the human body. The position data and the speed data are output to the data accumulation unit 14 and the receiving device control unit 18. The acceleration processing unit 15 and the speed/position detection unit 16 configure a capsule position detection unit which detects a position of the capsule endoscope 1 based on the image data and the acceleration data.

The data accumulation unit 14 (storage medium) accumulates the image data from the second image processing unit 13 and the position data and the speed data from the speed/position detection unit 16. The receiving device control unit 18 (receiving device control circuit) controls an operation of each unit. For example, the receiving device control unit 18 generates a frame rate designation value according to the speed data from the speed/position detection unit 16. The control data generation unit 17 (control data generation circuit) generates control data from the frame rate designation value from the receiving device control unit 18, and outputs the generated control data to the second wireless communication unit 12. The second image processing unit 13, the acceleration processing unit 15, the speed/position detection unit 16, the control data generation unit 17, and the receiving device control unit 18 may also be configured with an integrated circuit such as a processor. The second power supply unit 19 (second power supply circuit) provides each unit with power.

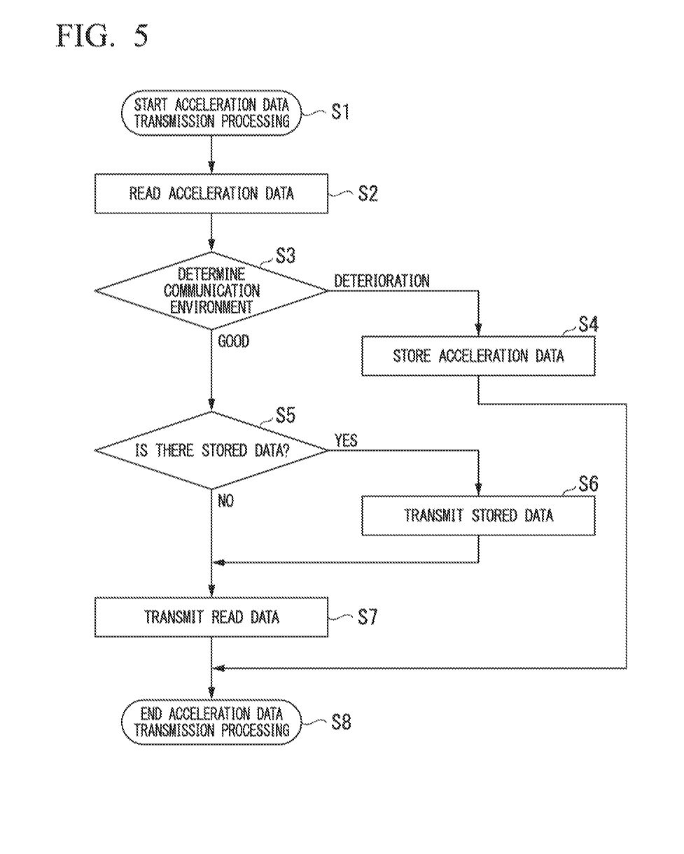

Acceleration data transmission processing performed by the capsule endoscope 1 will be described with reference to FIG. 5. FIG. 5 shows a procedure of the acceleration data transmission processing performed by the capsule endoscope 1.

The capsule control unit 10 performs the acceleration data transmission processing by controlling each unit in the capsule endoscope 1. The acceleration data transmission processing of the embodiments of the present invention is performed in synchronization with an imaging operation of the imaging unit 4. For example, when imaging is performed at 2 frames/second, the acceleration data transmission processing is performed at periods of an integral multiple of ½ second (0.5, 1, 2, 4 seconds, and the like). For example, as a period of the acceleration data transmission processing, it is possible to designate a period corresponding to a frame rate. Alternatively, the receiving device 2 separately transmits data of the period as the control data, and thereby it is possible to designate the period of the acceleration data transmission processing.

If the acceleration data transmission processing starts (S1), the capsule control unit 10 executes reading (S2) of the acceleration data. In the reading (S2) of the acceleration data, the capsule control unit 10 reads the acceleration data from the acceleration sensor 5. In the reading (S2) of the acceleration data, time data which represents a time at which the acceleration data is read is added to the acceleration data.

After the acceleration data is read, the capsule control unit 10 executes communication environment determination (S3). In the communication environment determination (S3), the capsule control unit 10 reads a result of the detection of the wireless communication environment from the communication environment detection unit 11, and decides processing according to the result of the detection of the wireless communication environment. That is, the capsule control unit 10 detects the wireless communication environment.

When the wireless communication environment deteriorates, the capsule control unit 10 performs storage of the acceleration data (S4). In the storage (S4) of the acceleration data, the capsule control unit 10 causes the acceleration data storage unit 6 to store the acceleration data to which the time data is added. When deterioration in the wireless communication environment is detected, the acceleration data storage unit 6 temporarily stores the acceleration data output from the acceleration sensor. After the storage (S4) of the acceleration data is executed, the acceleration data transmission processing ends (S8).

When the wireless communication environment is good, the capsule control unit 10 determines whether there is data stored in the storage (S4) of the acceleration data (S5). When there is stored data, the capsule control unit 10 executes transmission (S6) of the stored data. In the transmission (S6) of the stored data, the capsule control unit 10 transmits the acceleration data stored in the acceleration data storage unit 6 to the receiving device 2 by the first wireless communication unit 7. In other words, the first wireless communication unit 7 transmits the acceleration data stored in the storage (S4) of the acceleration data to the receiving device 2 by wireless communication after recovery of the wireless communication environment is detected.

Thereafter, the capsule control unit 10 executes transmission (S7) of read data. In the transmission (S7) of the read data, the capsule control unit 10 transmits the latest acceleration data read in the reading (S2) of the acceleration data to the receiving device 2 by the first wireless communication unit 7. That is, when deterioration in the wireless communication environment is not detected, the first wireless communication unit 7 transmits the latest acceleration data read in the reading (S2) of the acceleration data to the receiving device 2 by wireless communication. After the transmission of the read data (S7) is executed, the acceleration data transmission processing ends (S8).

When there is no stored data, the capsule control unit 10 executes the transmission (S7) of the read data. After the transmission (S7) of the read data is executed, the acceleration data transmission processing ends (S8).

At least one of the configurations other than the imaging unit 4, the acceleration sensor 5, the acceleration data storage unit 6, the first wireless communication unit 7, the communication environment detection unit 11, and the capsule control unit 10 may be omitted from the capsule endoscope in each embodiment of the present invention. Moreover, at least one of the configurations other than the second wireless communication unit 12, the acceleration processing unit 15, and the speed/position detection unit 16 may be omitted from the receiving device in each embodiment of the present invention.

According to the first embodiment, the capsule endoscope system 100 is configured to have the capsule endoscope 1 and the receiving device 2. The capsule endoscope 1 includes the imaging unit 4, the acceleration sensor 5, the acceleration data storage unit 6, the first wireless communication unit 7, the communication environment detection unit 11, and the capsule control unit 10. The receiving device 2 includes the second wireless communication unit 12 and the capsule position detection unit (the acceleration processing unit 15, the speed/position detection unit 16).

In addition, according to the first embodiment, the capsule endoscope 1 is configured to have the imaging unit 4, the acceleration sensor 5, the acceleration data storage unit 6, the first wireless communication unit 7, the communication environment detection unit 11, and the capsule control unit 10.

Moreover, according to the first embodiment, a first wireless communication method of the capsule endoscope 1 is configured to have the communication environment detection step (S3), the storage step (S4), and the transmission step (S6).

In addition, according to the first embodiment, a program is configured to cause a computer of the capsule endoscope 1 to execute the communication environment detection step (S3), the storage step (S4), and the transmission step (S6).

According to the first embodiment, when deterioration in the wireless communication environment is detected, after the acceleration data is temporarily stored and then recovery of the wireless communication environment is detected, the stored acceleration data is transmitted from the capsule endoscope 1. Therefore, the receiving device 2 may acquire acceleration data when the wireless communication environment deteriorates. As a result, when the wireless communication environment deteriorates, it is possible to suppress degradation of accuracy in position detection of the capsule endoscope 1.

### Second Embodiment

A capsule endoscope of a second embodiment of the present invention has the functions of the capsule endoscope 1 shown in the first embodiment. In addition, the capsule endoscope of the second embodiment has a function of treatment work including medication or “collection of tissues or body fluid,” and an execution control function of the treatment work. The receiving device of the second embodiment has a repeater and an operation/saving device.

The repeater is attached to a patient. The repeater is mainly responsible for wireless communication with a capsule endoscope. The operation/saving device is disposed separately from the repeater. The operation/saving device operates while wirelessly connected to the repeater, and is responsible for saving of the image data and control of the treatment work.

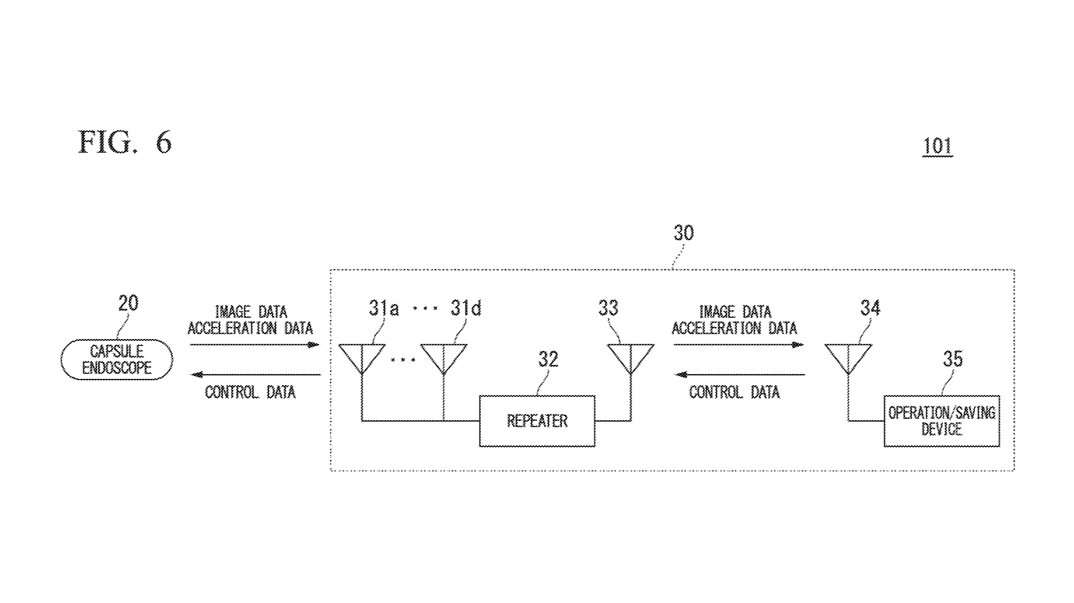

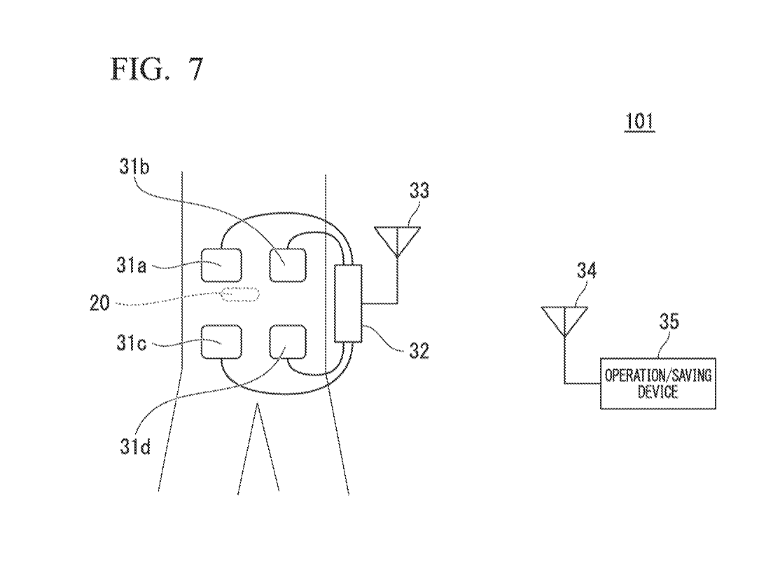

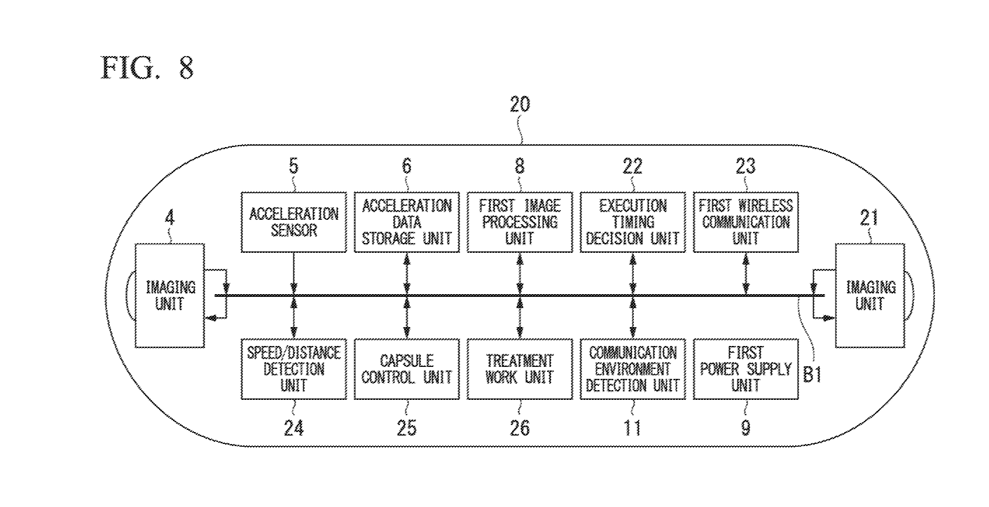

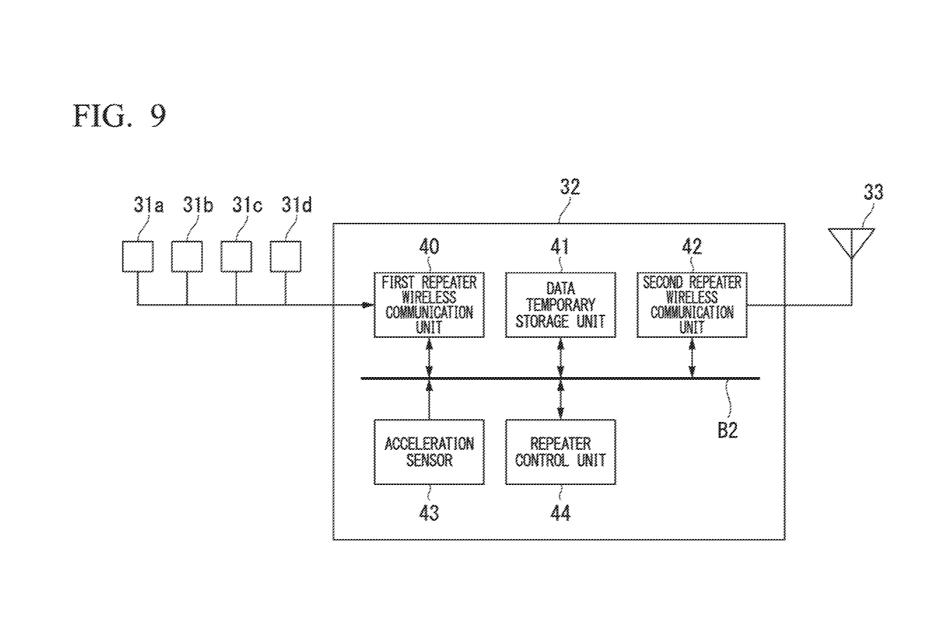

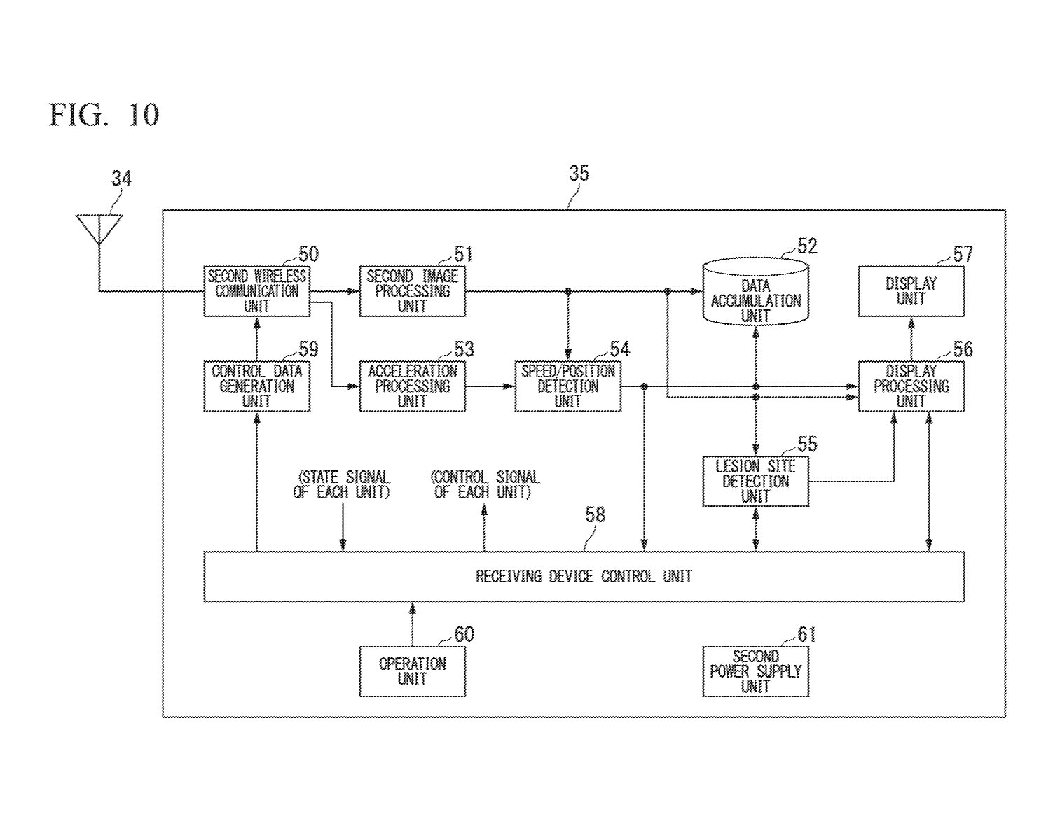

A system configuration, a device configuration, and an outline of operations will be described with reference to FIGS. 6 to 10. FIG. 6 shows a configuration of a capsule endoscope system 101. FIG. 7 shows a usage state of the capsule endoscope system 101. FIG. 8 shows a configuration of a capsule endoscope 20. FIG. 9 shows a configuration of a repeater 32. FIG. 10 shows a configuration of an operation/saving device 35.

As shown in FIG. 6, the capsule endoscope system 101 of the second embodiment includes the capsule endoscope 20 and a receiving device 30. The receiving device 30 includes the repeater 32 and the operation/saving device 35.

FIG. 7 shows a state in which the antennas 31a to 31d are attached to a human body (patient) and a positional relationship among the capsule endoscope 20, the repeater 32, and the operation/saving device 35. As described above, the capsule endoscope 20 has a treatment function and executes the treatment work according to an instruction from the operation/saving device 35. In the second embodiment, the image data and the acceleration data are transmitted to the operation/saving device 35 from the capsule endoscope 20 via the repeater 32. The image data and the acceleration data are saved in the operation/saving device 35. In the second embodiment, acceleration of the capsule endoscope 20 and acceleration of the repeater 32 are measured. Acceleration data of the repeater 32 is transmitted to the operation/saving device 35 separately from acceleration data of the capsule endoscope 20. The operation/saving device 35 subtracts the acceleration data of the repeater 32 from the acceleration data of the capsule endoscope 20. As a result, acceleration data excluding acceleration data generated by movement of the human body is obtained. Accordingly, it is possible to more accurately calculate a position of the capsule endoscope 20.

Control data of the second embodiment is one of frame rate control data, work execution condition data, and work instruction data in the same manner as in the first embodiment. The work execution condition data represents a position at which the treatment work is performed. The work instruction data represents an execution instruction of the treatment work. The control data is generated by the operation/saving device 35. The generated control data is transmitted to the capsule endoscope 20 via the repeater 32. Details of the work execution condition data and the work instruction data will be described below.

Wireless communication between the capsule endoscope 20 and the repeater 32 is performed through an antenna in the capsule endoscope 20 and antennas 31a to 31d of the repeater 32. Wireless communication between the repeater 32 and the operation/saving device 35 is performed through an antenna 33 of the repeater 32 and an antenna 34 of the operation/saving device 35. In FIG. 6, only the antenna 31a, the antenna 31d, the antenna 33 and the antenna 34 are shown.

As shown in FIG. 8, the capsule endoscope 20 includes the imaging unit 4, the acceleration sensor 5, the acceleration data storage unit 6, the first image processing unit 8, the first power supply unit 9, the communication environment detection unit 11, and the data bus B1. Furthermore, the capsule endoscope 20 includes an imaging unit 21, an execution timing decision unit 22, a first wireless communication unit 23, a speed/distance detection unit 24, a capsule control unit 25, and a treatment work unit 26.

Differences of the configuration shown in FIG. 8 from the configuration shown in FIG. 3 will be described. The imaging unit 4 (imaging element) and the imaging unit 21 (imaging element) perform imaging and output image data. The imaging unit 4 and the imaging unit 21 perform imaging in a human body at a designated frame rate. The imaging unit 4 and the imaging unit 21 are disposed at both ends (a first end and a second end different from the first end) of a main body of the capsule endoscope 20 so that respective imaging surfaces face away from each other. The imaging unit 4 is disposed at the first end of the capsule endoscope 20 so that an imaging direction is an outer direction. The imaging unit 21 is disposed at the second end of the capsule endoscope 20 so that an imaging direction is an outer direction, that is, a direction substantially opposite to the imaging direction of the imaging unit 4. In addition, the imaging unit 4 and the imaging unit 21 are disposed so that the imaging directions are substantially the same as a movement direction of the capsule endoscope 20 or an opposite direction thereto.

When the treatment work is performed, among the imaging unit 4 and the imaging unit 21, the imaging unit capable of imaging a lesion site is selected by the receiving device 30. When the treatment work is performed, only the selected imaging unit performs imaging. When the treatment work is not performed, the imaging unit 4 and the imaging unit 21 alternately perform imaging.

The first wireless communication unit 23 (first wireless communication circuit) performs the same wireless communication as the wireless communication performed by the first wireless communication unit 7 in the first embodiment. The first wireless communication unit 23 further receives the work execution condition data and the work instruction data from the receiving device 30. As described above, the work execution condition data represents a position at which the treatment work is performed. The work instruction data represents an execution instruction of the treatment work.

The speed/distance detection unit 24 (speed/distance detection circuit) detects a movement speed and a movement distance of the capsule endoscope 20 based on the acceleration data from the acceleration sensor 5. The execution timing decision unit 22 instructs output of an execution command at a timing based on the movement distance detected by the speed/distance detection unit 24 and the work execution condition data received by the first wireless communication unit 23. As a result, the execution timing decision unit 22 decides a timing independent from a timing designated by the work instruction data from the receiving device 30. The execution timing decision unit 22 notifies the capsule control unit 25 of a decided timing by instructing the capsule control unit 25 to output an execution command. The treatment work unit 26 performs medication or “collection of tissue or body fluid” based on the execution command from the capsule control unit 25. That is, the treatment work unit 26 performs medication. Alternatively, the treatment work unit 26 performs collection of tissue or body fluid.

The capsule control unit 25 (capsule control circuit) performs the same control as the control performed by the capsule control unit 10 in the first embodiment. The capsule control unit 25 further performs control of the treatment work. For example, the capsule control unit 25 decides a timing for the treatment work and controls execution of the treatment work at the decided timing based on the movement speed of the capsule endoscope 20 and a wireless communication environment after the work execution condition data is received. The timing for the treatment work is one of a timing instructed by the receiving device 30 and a timing instructed by the execution timing decision unit 22. Specifically, when the movement speed is low, the capsule control unit 25 outputs an execution command to the treatment work unit 26 at the timing at which the work instruction data is received. When the movement speed is high, the capsule control unit 25 outputs an execution command to the treatment work unit 26 at the timing at which output of the execution command is instructed by the execution timing decision unit 22.

When the movement speed is low and deterioration in the wireless communication environment is not detected by the communication environment detection unit 11, the capsule control unit 25 further outputs an execution command to the treatment work unit 26 at the timing at which the work instruction data is received. When the movement speed is low and deterioration in the wireless communication environment is detected by the communication environment detection unit 11, the capsule control unit 25 further outputs an execution command to the treatment work unit 26 at the timing at which output of the execution command is instructed by the execution timing decision unit 22.

The first image processing unit 8, the communication environment detection unit 11, the execution timing decision unit 22, the speed/distance detection unit 24, and the capsule control unit 25 may be configured with an integrated circuit such as a processor. Except for what has been described above, the configuration shown in FIG. 8 is the same as the configuration shown in FIG. 3.

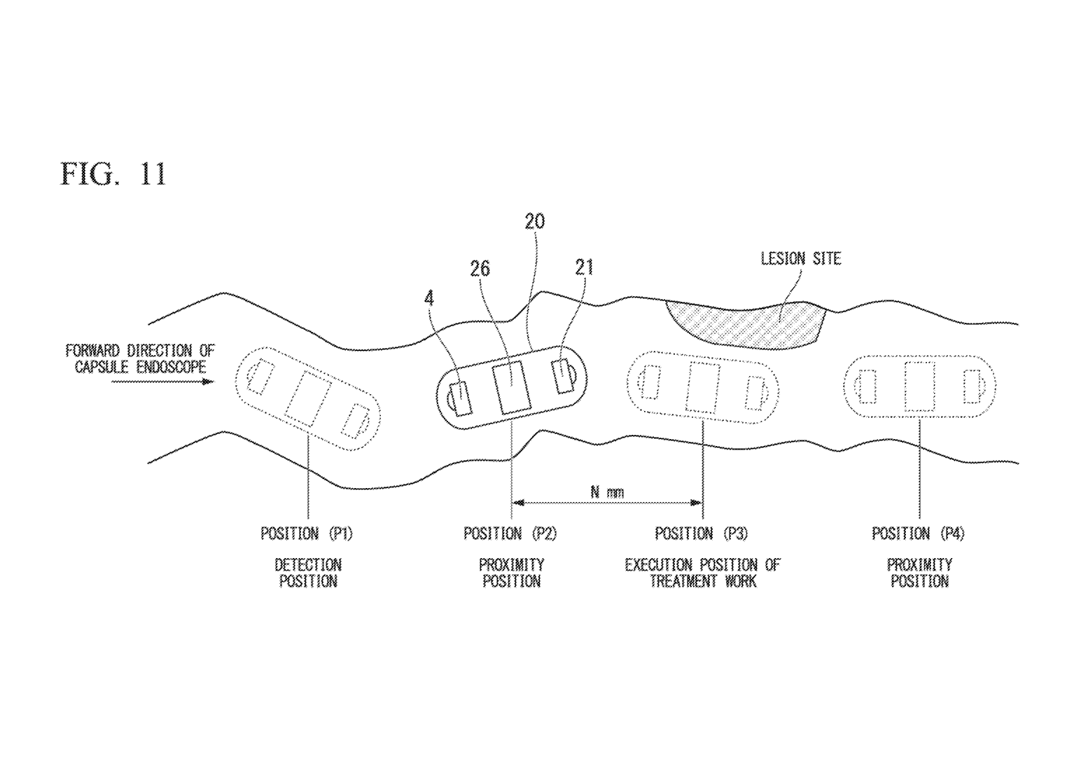

An operator recognizes an image of the lesion site displayed on the operation/saving device 35, and decides an execution condition of the treatment work. Work execution condition data generated according to a decided execution condition is transferred to the capsule control unit 25 of the capsule endoscope 20 through the repeater 32. A method of deciding an execution timing of the treatment work will be described in detail below using FIGS. 11 to 14.

The repeater 32 configuring the receiving device 30 is attached to the body of a patient. The repeater 32 performs relay of data transfer between the capsule endoscope 20 and the operation/saving device 35. As shown in FIG. 9, the repeater 32 includes the antennas 31a, 31b, 31c, 31d, and 33, a first repeater wireless communication unit 40 (second wireless communication unit), a data temporary storage unit 41, a second repeater wireless communication unit 42, an acceleration sensor 43, a repeater control unit 44, and a data bus B2.

The antennas 31a, 31b, 31c, and 31d are wirelessly connected to the capsule endoscope 20. The first repeater wireless communication unit 40 (first repeater wireless communication circuit) receives the image data and the acceleration data from the capsule endoscope 20 by wireless communication. In addition, the first repeater wireless communication unit 40 transmits control data to the capsule endoscope 20 by the wireless communication. The control data of the second embodiment may be the work execution condition data or the work instruction data. The work execution condition data and the work instruction data are generated by the operation/saving device 35. Therefore, the first repeater wireless communication unit 40 transmits the work execution condition data and the work instruction data generated by the operation/saving device 35 to the capsule endoscope 20.

The repeater 32 is equipped with the data temporary storage unit 41 (storage medium) to cope with communication failure occurring during relay processing. The second repeater wireless communication unit 42 (second repeater wireless communication circuit) transmits the image data and the acceleration data by wireless communication to the operation/saving device 35. Moreover, the second repeater wireless communication unit 42 receives the control data from the operation/saving device 35 by the wireless communication. The control data of the second embodiment may be the work execution condition data or the work instruction data. Therefore, the second repeater wireless communication unit 42 receives the work execution condition data and the work instruction data from the operation/saving device 35.

In order to detect acceleration in accordance with movement of a patient, the repeater 32 is equipped with the acceleration sensor 43. The acceleration sensor 43 detects acceleration applied to the repeater 32 and outputs acceleration data. The acceleration data from the acceleration sensor 43 is transmitted to the operation/saving device 35. The repeater control unit 44 controls an operation of each unit. The data bus B2 performs transfer of various types of data.

The operation/saving device 35 configuring the receiving device 30 performs saving of the image data and saving of the position data of the capsule endoscope 20 calculated based on the acceleration data in the same manner as the receiving device 2 of the first embodiment. In addition, unlike the first embodiment, the operation/saving device 35 has a display function for an operator to perform the treatment work and a control function for the treatment work. As shown in FIG. 10, the operation/saving device 35 includes the antenna 34, a second wireless communication unit 50, a second image processing unit 51, a data accumulation unit 52, an acceleration processing unit 53, a speed/position detection unit 54, a lesion site detection unit 55, a display processing unit 56, a display unit 57, a receiving device control unit 58 (generation unit), a control data generation unit 59, an operation unit 60, and a second power supply unit 61.

The antenna 34 is wirelessly connected to the repeater 32. The second wireless communication unit 50 (second wireless communication circuit) receives the image data and the acceleration data from the repeater 32 by wireless communication. In addition, the second wireless communication unit 50 transmits control data to the repeater 32 by wireless communication. The second image processing unit 51 (second image processing circuit) is the same as the second image processing unit 13 in the first embodiment.

The acceleration processing unit 53 (acceleration processing circuit) converts the acceleration data received by the second wireless communication unit 50 into speed data and movement distance data at predetermined time intervals. At this time, the acceleration processing unit 53 subtracts the acceleration data of the repeater 32 from the acceleration data of the capsule endoscope 20, thereby obtaining acceleration data excluding acceleration data generated by the movement of a patient. The speed data and the movement distance data are output to the speed/position detection unit 54. The speed/position detection unit 54 (speed/position detection circuit) is the same as the speed/position detection unit 16 in the first embodiment. The data accumulation unit 52 (storage medium) is the same as the data accumulation unit 14 in the first embodiment.

The lesion site detection unit 55 detects a lesion site based on the image data. Various algorithms for detecting a lesion site based on image data have been devised. Since these algorithms are well-known, detailed descriptions thereof will be omitted. Positional information of the lesion site detected by the lesion site detection unit 55 is output to the display processing unit 56. The display processing unit 56 superimposes information based on the positional information of the lesion site onto the image data from the second image processing unit 51. The image data processed by the display processing unit 56 is output to the display unit 57. The display unit 57 displays images based on the image data.

The operation unit 60 receives operations of an operator. The receiving device control unit 58 (receiving device control circuit) performs the same control as the control performed by the receiving device control unit 18 in the first embodiment. The receiving device control unit 58 further generates work execution condition data and work instruction data based on the operations received by the operation unit 60. The work execution condition data and the work instruction data are output to the control data generation unit 59. The control data generation unit 59 (control data generation circuit) generates control data from a frame rate instruction value, the work execution condition data, and the work instruction data from the receiving device control unit 58, and outputs the generated control data to the second wireless communication unit 50. As described above, the second wireless communication unit 50 transmits the control data to the repeater 32. Accordingly, the second wireless communication unit 50 transmits the work execution condition data and the work instruction data generated by the receiving device control unit 58 to the repeater 32.

The second image processing unit 51, the acceleration processing unit 53, the speed/position detection unit 54, the lesion site detection unit 55, the display processing unit 56, the receiving device control unit 58, and the control data generation unit 59 may be configured with an integrated circuit such as a processor. The second power supply unit 61 (second power supply circuit) supplies power to each unit.

Hereinafter, an operation of the operation/saving device 35 will be described focusing on a display function and a control function for the treatment work. The operator decides a position at which the treatment work for a lesion site is performed while observing an image displayed on the display unit 57. The receiving device control unit 58 is notified of the decided position through the operation unit 60.

“A position at which the treatment work is performed” decided by the operator at this time is not a position based on an instruction from the receiving device 30. The position at which to perform the treatment work decided by the operator at this time is a position at which the capsule endoscope 20 performs the treatment work independently. When the capsule endoscope 20 passes through a lesion site at a high movement speed or deterioration in the wireless communication environment occurs, the capsule endoscope 20 performs the treatment work independently. When the capsule endoscope 20 moves at a high speed, there is a possibility that the treatment work is performed at a position different from the position based on the instruction from the receiving device 30. Moreover, when the wireless communication environment deteriorates, there is a possibility that the capsule endoscope 20 is not notified of the instruction from the receiving device 30. For this reason, the capsule endoscope 20 is capable of performing the treatment work independently.

For example, when the treatment work for the lesion site is medication, the capsule endoscope 20 performs the medication immediately before the capsule endoscope 20 reaches the lesion site. Medicated medicine reaches the lesion site as time elapses. When a position of the medication is very far from the lesion site, there is a possibility that the medicine widely spreads and the medicine is applied too thin. In addition, when the position of the medication is very close to the lesion site, there is a possibility that the medicine is not applied to a portion of the lesion site.

Therefore, the operator decides an execution position of the medication in consideration of a shape of the lesion site and a nature of the medicine. Specifically, the operator decides “a position at which the treatment work is performed” while observing an image displayed on the display unit 57. The operator operates the operation unit 60 and inputs “the position at which the treatment work is performed.” The receiving device control unit 58 decides a work execution condition based on a relationship between the decided “position at which the treatment work is performed” and a position of the lesion site, and generates the work execution condition data.

The work execution condition data includes work content information representing the content of the treatment work such as medication and body fluid collection and includes work execution position information representing a positional relationship between the lesion site and the capsule endoscope 20 when the capsule endoscope 20 independently executes the treatment work. When a treatment function of the capsule endoscope 20 is medication, the content of the treatment work is “medication execution.” For example, a work execution position is “a position at which the capsule endoscope 20 moves 20 mm from the position at which the work execution condition data is received.”

An example in which the treatment function of the capsule endoscope 20 is body fluid sampling in which the body fluid collection is performed in a periphery of the lesion site will be described. The body fluid collection is performed when the capsule endoscope 20 is on the lesion site or immediately after the capsule endoscope 20 passes through the lesion site. Therefore, the content of the treatment work is “body fluid sampling execution.” For example, the work execution position is “a position at which the capsule endoscope 20 moves 30 mm from the position at which the work execution condition data is received.”

The work execution condition data generated by the receiving device control unit 58 is transmitted to the repeater 32 through the control data generation unit 59 and the second wireless communication unit 50. The work execution condition data received by the repeater 32 is transmitted to the capsule endoscope 20 by the repeater 32.

In the second embodiment, when the capsule endoscope 20 is positioned a predetermined distance from the lesion site, the receiving device 30 transmits the work execution condition data to the capsule endoscope 20. The capsule endoscope 20 executes the treatment work when the capsule endoscope 20 moves a distance designated in the work execution condition data from the position at which the work execution condition data is received.

When the capsule endoscope 20 passes through the lesion site at a high movement speed and deterioration in the wireless communication environment does not occur, the operator decides a timing at which to perform the treatment work while observing an image displayed on the display unit 57. The operator operates the operation unit 60 and inputs an instruction of the treatment work at the timing at which the treatment work is performed. The receiving device control unit 58 generates the work instruction data based on the instruction of the treatment work.

The work instruction data generated by the receiving device control unit 58 is transmitted to the repeater 32 through the control data generation unit 59 and the second wireless communication unit 50. The work instruction data received by the repeater 32 is transmitted to the capsule endoscope 20 by the repeater 32. The capsule endoscope 20 executes the treatment work at the timing at which the work instruction data is received. A generation method of the work execution condition data and the work instruction data will be described in detail referring to FIGS. 11 to 13.

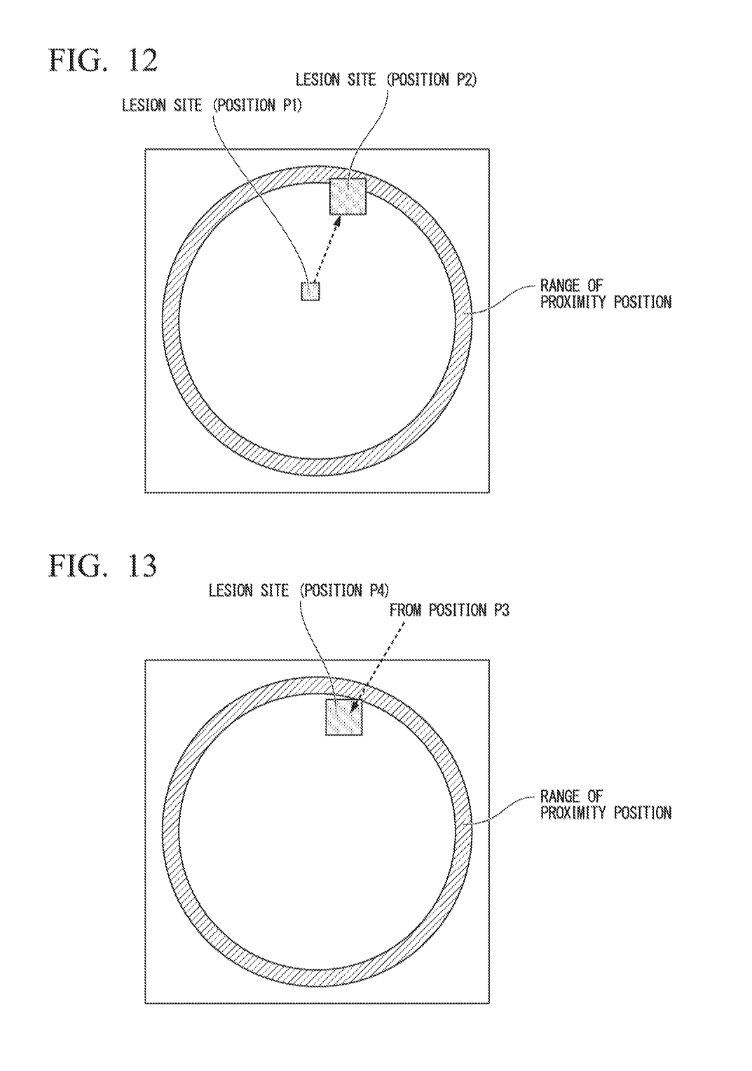

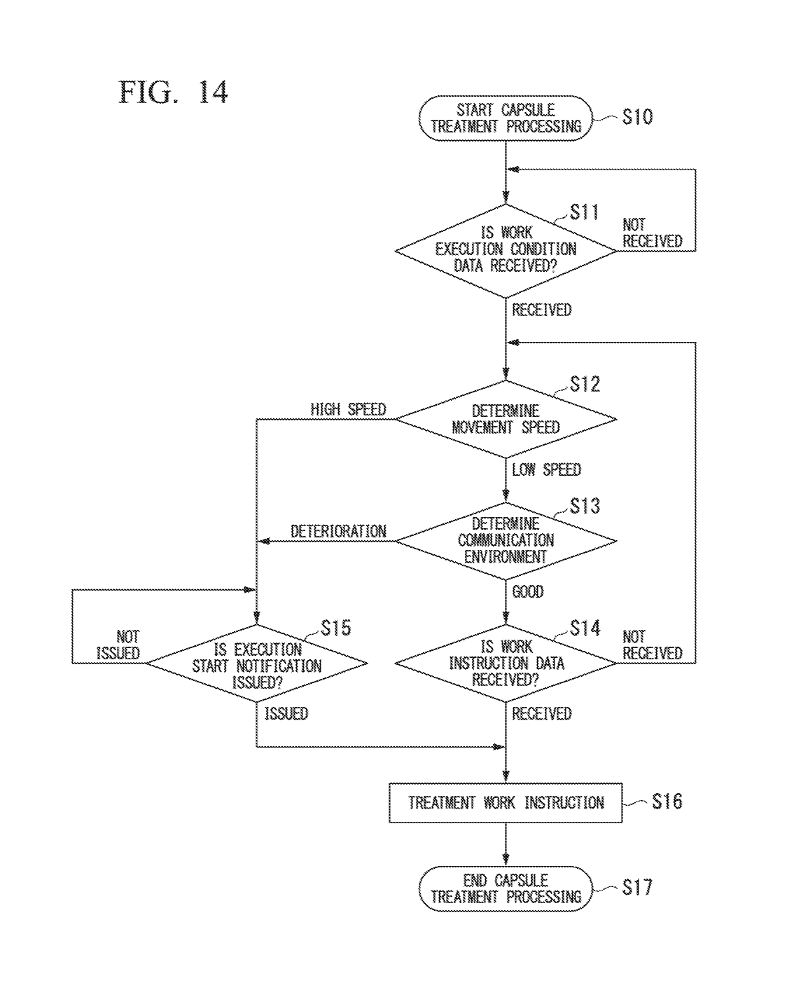

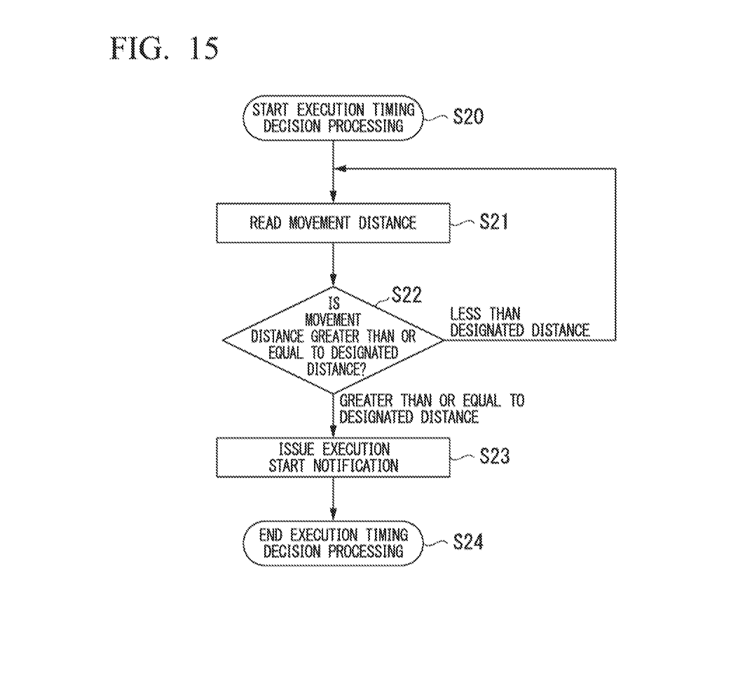

The treatment work will be described in detail referring to FIGS. 11 to 15. FIG. 11 shows a state of the capsule endoscope 20 when the capsule endoscope 20 performs the treatment work. FIG. 12 shows an image captured by an imaging unit at the front (in a forward direction) of the capsule endoscope 20 during the treatment work. FIG. 13 shows an image captured by an imaging unit at the rear (in a backward direction) of the capsule endoscope 20 during the treatment work. FIG. 14 shows a procedure of treatment work processing performed by the capsule endoscope 20. FIG. 15 shows a procedure of execution timing decision processing performed by the capsule endoscope 20.

FIG. 11 shows a position of the capsule endoscope 20 in an intestinal tract and an execution timing of the treatment work for the lesion site. In FIG. 11, the capsule endoscope 20 moves to the right. The capsule endoscope 20 detects the lesion site at a position (P1). The capsule endoscope 20 receives the work execution condition data at a proximity position (P2). The capsule endoscope 20 performs the treatment work (medication) at an execution position (P3) of the treatment work. The capsule endoscope 20 performs imaging in the backward direction to confirm the execution of the treatment work until the capsule endoscope 20 reaches a proximity position (P4) in the opposite direction.

For example, when the capsule endoscope 20 passes through a 6 m long small intestine in 3 hours, an average speed of the capsule endoscope 20 is 0.56 mm/s (6000/(3×60×60)=0.56). For example, when a total length of the capsule endoscope 20 is about 26 mm, it takes about 46 seconds for the capsule endoscope 20 to move a distance of the total length. For example, when a distance N between the position (P2) and the position (P3) shown in FIG. 11 is 30 mm which is even longer than the total length of the capsule endoscope 20, it takes about 53 seconds for the capsule endoscope 20 to move from the position (P2) to the position (P3). When a frame rate is 2 frames/second, 106 images are captured during the movement from the position (P2) to the position (P3). Therefore, even when the operator operates while observing the images, it is possible to sufficiently maintain accuracy of the treatment work.

FIG. 12 and FIG. 13 are examples of images captured by the capsule endoscope 20 during the treatment work. FIG. 12 shows an image captured by the imaging unit 21 facing the forward direction of the capsule endoscope 20 at the position (P1) and the position (P2) of FIG. 11. In FIG. 12, the lesion site is shown in a rectangular shape.

When the capsule endoscope 20 is at the position (P1), the lesion site is far from the capsule endoscope 20. For this reason, there is a small lesion site at the center of the image. When the capsule endoscope 20 is at the position (P2), the lesion site is close to the capsule endoscope 20. For this reason, there is a large lesion site in the periphery of the image. In the second embodiment, when the capsule endoscope 20 is positioned between the position (P1) and the position (P2), the operator decides a work execution condition for the lesion site. When the capsule endoscope 20 reaches the position (P2), the work execution condition data is transmitted to the capsule endoscope 20 from the operation/saving device 35 through the repeater 32.

More specifically, a range between two circles in FIG. 12 is a range of the proximity position. Approximation of the capsule endoscope 20 to the lesion site beyond the position (P2) is detected from images in the range of the proximity position. When the speed/position detection unit 54 of the operation/saving device 35 detects the lesion site from the range of the proximity position, the receiving device control unit 58 transmits the work execution condition data to the repeater 32 using the second wireless communication unit 50.

The treatment work will be described in detail below using a specific example of timing. For example, in FIG. 11, when the distance N between the position (P2) and the position (P3) is 30 mm, the content of the work execution condition of which the capsule endoscope 20 is notified represents that the treatment work is executed at a position at which the capsule endoscope 20 moves 30 mm forward from the position (P2) at which the work execution condition data is received. After the treatment work is executed, imaging in the backward direction is performed until the capsule endoscope 20 reaches the position (P4) to confirm a result of the execution.

FIG. 13 shows an image captured by the imaging unit 4 facing the backward direction of the capsule endoscope 20. A range between two circles in FIG. 13 is a range of the proximity position. Separation of the capsule endoscope 20 from the lesion site beyond the position (P4) is detected from images in the range of the proximity position. When the speed/position detection unit 54 of the operation/saving device 35 detects that the lesion site is out of the range of the proximity position, the receiving device control unit 58 transmits control data representing an end of imaging to the repeater 32 using the second wireless communication unit 50.

As described above, independent treatment work by the capsule endoscope 20 based on the work execution condition data is performed only when the capsule endoscope 20 moves at a high speed after the work execution condition data is received, or when a wireless communication environment between the repeater 32 and the capsule endoscope 20 deteriorates.

Capsule treatment processing performed by the capsule endoscope 20 will be described using FIG. 14. FIG. 14 shows a procedure of the capsule treatment processing performed by the capsule endoscope 20. The capsule control unit 25 performs the capsule treatment process by controlling each unit in the capsule endoscope 20.

When the capsule treatment processing starts (S10), the capsule control unit 25 executes reception determination (S11) of the work execution condition data. In the reception determination (S11) of the work execution condition data, the capsule control unit 25 determines whether the work execution condition data is received. When the work execution condition data is not received, the reception determination (S11) of the work execution condition data is repeated.

The first wireless communication unit 23 receives the work execution condition data from the receiving device 30. When the work execution condition data is received, the capsule control unit 25 executes movement speed determination (S12) based on the speed data from the speed/distance detection unit 24. In the movement speed determination (S12), the capsule control unit 25 determines a movement speed of the capsule endoscope 20. For example, the capsule control unit 25 determines whether the movement speed of the capsule endoscope 20 is higher than or equal to a predetermined speed. When the movement speed of the capsule endoscope 20 is higher than or equal to the predetermined speed, the capsule control unit 25 determines that the movement speed of the capsule endoscope 20 is high. In addition, when the movement speed of the capsule endoscope 20 is lower than the predetermined speed, the capsule control unit 25 determines that the movement speed of the capsule endoscope 20 is low.

When the movement speed of the capsule endoscope 20 is low, the capsule control unit 25 executes communication environment determination (S13). In the communication environment determination (S13), the capsule control unit 25 reads a result of the detection of a wireless communication environment from the communication environment detection unit 11 and decides processing according to the result of the detection of a wireless communication environment. That is, the capsule control unit 25 detects a wireless communication environment.

When the wireless communication environment is good, the capsule control unit 25 executes reception determination (S14) of the work instruction data. In the reception determination (S14) of the work instruction data, the capsule control unit 25 determines whether the work instruction data is received. When the work instruction data is not received, the capsule control unit 25 executes the movement speed determination (S12).

The first wireless communication unit 23 receives the work instruction data from the receiving device 30. When the work instruction data is received, the capsule control unit 25 executes treatment work instruction (S16). In the treatment work instruction (S16), the capsule control unit 25 outputs an execution command for treatment work to the treatment work unit 26. That is, the capsule control unit 25 outputs the execution command to the treatment work unit 26 at a timing at which the work instruction data is received when the movement speed is low and deterioration in the wireless communication environment is not detected by the communication environment detection unit 11. The treatment work unit 26 executes the treatment work based on the execution command from the capsule control unit 25. After the treatment work instruction (S16) is executed, the capsule treatment processing ends (S17).

When the movement speed of the capsule endoscope 20 is high, or when the wireless communication environment deteriorates, the capsule control unit 25 executes issuance determination (S15) of an execution start notification. In the issuance determination (S15) of an execution start notification, the capsule control unit 25 determines whether an execution start notification is issued from the execution timing decision unit 22. By issuance of the execution start notification, output of the execution command for the treatment work is instructed. When the execution start notification is not issued, the issuance determination (S15) of an execution start notification is repeated.

When the execution start notification is issued, the capsule control unit 25 executes the treatment work instruction (S16). That is, the capsule control unit 25 outputs an execution command to the treatment work unit 26 at a timing at which output of the execution command is instructed by the execution timing decision unit 22 when the movement speed is high. In addition, the capsule control unit 25 outputs the execution command to the treatment work unit 26 at the timing at which output of the execution command is instructed by the execution timing decision unit 22 when the movement speed is low and deterioration in the wireless communication environment is detected by the communication environment detection unit 11. The treatment work unit 26 executes the treatment work based on the execution command from the capsule control unit 25. After the treatment work instruction (S16) is executed, the capsule treatment processing ends (S17).

When the movement speed of the capsule endoscope 20 is low and the wireless communication environment is good, the process of S12, S13, and S14 are repeated. These kinds of processing are determination processing, and are performed at a high speed. Therefore, a delay time from reception of the work instruction data to execution of the treatment work can be ignored.

The execution timing decision processing performed by the capsule endoscope 20 will be described using FIG. 15. FIG. 15 shows a procedure of the execution timing decision processing performed by the capsule endoscope 20. The execution timing decision unit 22 performs the execution timing decision processing.

The execution timing decision unit 22 executes the execution timing decision processing shown in FIG. 15 immediately after the work execution condition data is received from the receiving device 30. When the execution timing decision processing starts (S20), the execution timing decision unit 22 executes reading (S21) of a movement distance. In the reading (S21) of a movement distance, the execution timing decision unit 22 reads the movement distance data from the speed/distance detection unit 24.

After the movement distance data is read, the execution timing decision unit 22 executes determination of a movement distance (S22). In the determination of a movement distance (S22), the execution timing decision unit 22 determines whether a movement distance represented by the movement distance data is greater than or equal to the distance designated by the work execution condition data. When the movement distance is less than the distance designated by the work execution condition data, the execution timing decision unit 22 executes the reading (S21) of a movement distance.

When the movement distance is greater than or equal to the distance designated by the work execution condition data, the execution timing decision unit 22 executes issuance (S23) of an execution start notification. In the issuance (S23) of an execution start notification, the execution timing decision unit 22 issues an execution start notification to the capsule control unit 25. Accordingly, the execution timing decision unit 22 instructs the capsule control unit 25 to output an execution command for the treatment work. After the issuance (S23) of an execution start notification is executed, the execution timing decision processing ends (S24).

In the execution timing decision processing shown in FIG. 15, the execution timing decision unit 22 determines whether a movement distance from a timing at which the work execution condition data is received reaches the distance designated by the work execution position information included in the work execution condition data. Accordingly, the execution timing decision unit 22 decides an execution timing of the treatment work.

For example, the issuance (S23) of an execution start notification is executed at a timing at which the movement distance of the capsule endoscope 20 is determined to reach the movement distance (N=30 mm) designated by the work execution condition data. Accordingly, the treatment work instruction (S16) is executed. In the example shown in FIG. 11, after the capsule treatment processing ends, the capsule control unit 25 instructs the imaging unit 4 to perform imaging. Accordingly, imaging of the image shown in FIG. 13 is performed.

A decision on the execution timing of the treatment work may not depend on determination of the wireless communication environment. For example, when the movement speed is determined to be low in the movement speed determination (S12), the reception determination (S14) of the work instruction data may also be executed without performing the communication environment determination (S13). When the work instruction data is received, the treatment work instruction (S16) is executed. When the movement speed is low and deterioration in the wireless communication environment occurs, the processing of S12 and S14 are repeated. In this case, the operator observes the display unit 57. Accordingly, the operator confirms whether the capsule endoscope 20 has passed through the lesion site without performing the treatment work. When it is confirmed that the capsule endoscope 20 has passed through the lesion site, an additional instruction is output from the receiving device 30 and the capsule treatment processing stops.

In the second embodiment, the receiving device 30 is divided into the repeater 32 and the operation/saving device 35. However, a receiving device in which the repeater 32 and the operation/saving device 35 are integrated may be attached to a human body (patient).

In the second embodiment, when the movement speed of the capsule endoscope 20 is high, or when deterioration in the wireless communication environment is detected, the treatment work is executed at the timing instructed by the execution timing decision unit 22. For this reason, the capsule endoscope 20 can execute the treatment work at an appropriate timing.

### Third Embodiment

In a capsule endoscope system of a third embodiment of the present invention, the capsule endoscope 20 in the capsule endoscope system 101 shown in FIG. 6 is changed. A capsule endoscope of the third embodiment has the functions of the capsule endoscope 20. In addition, the capsule endoscope of the third embodiment has a function of imaging in the periphery of the lesion site at which the treatment work is performed and a function of temporarily storing image data in the capsule endoscope. Accordingly, the capsule endoscope can reliably transfer the image data to the receiving device regardless of deterioration in the wireless communication environment.

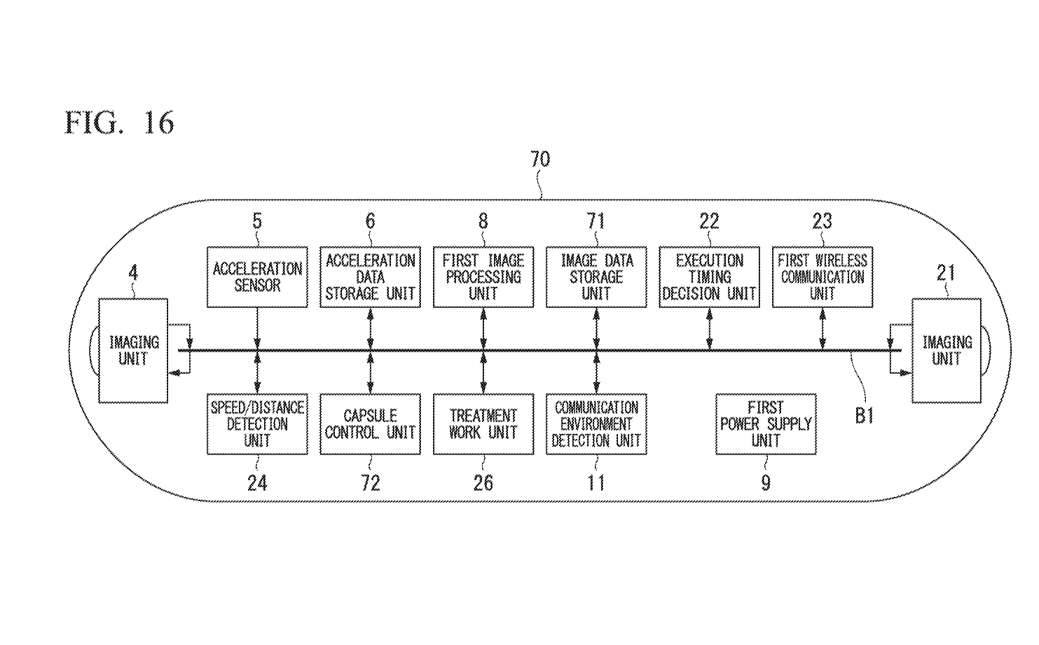

With reference to FIG. 16, a configuration and an operation outline of a capsule endoscope 70 of the third embodiment will be described. FIG. 16 shows the configuration of the capsule endoscope 70.

As shown in FIG. 16, the capsule endoscope 70 includes an imaging unit 4, an acceleration sensor 5, an acceleration data storage unit 6, a first image processing unit 8, a first power supply unit 9, a communication environment detection unit 11, an imaging unit 21, an execution timing decision unit 22, a first wireless communication unit 23, a speed/distance detection unit 24, a treatment work unit 26, and a data bus B1. Furthermore, the capsule endoscope 70 includes an image data storage unit 71 and a capsule control unit 72.

Differences of the configuration shown in FIG. 16 from the configuration shown in FIG. 8 will be described. The imaging unit 4 and the imaging unit 21 perform imaging according to a movement distance of the capsule endoscope 70 at a position based on a position at which an execution command for the treatment work is output. That is, the imaging unit 4 and the imaging unit 21 perform imaging according to a movement distance of the capsule endoscope 70 in the vicinity of the position at which the execution command for the treatment work is output. Therefore, the imaging unit 4 and the imaging unit 21 perform imaging at a position in the vicinity of the lesion site and output image data of the periphery of the lesion site.

The image data storage unit 71 (storage medium) temporarily stores the image data output from the imaging unit 4 and the imaging unit 21. The image data stored in the image data storage unit 71 is image data of the periphery of the lesion site at which the treatment work is performed. For example, the image data storage unit 71 is a storage medium different from the acceleration data storage unit 6. Alternatively, one storage medium may have a first storage region and a second storage region, the first storage region may be the acceleration data storage unit 6, and the second storage region may be the image data storage unit 71.

The capsule control unit 72 performs the same control as the capsule control unit 25 of the second embodiment. The capsule control unit 72 further has a control function of the image data storage unit 71 and a transmission function to transmit the image data stored in the image data storage unit 71 to the receiving device 30. Therefore, the first wireless communication unit 23 transmits the image data stored in the image data storage unit 71 to the receiving device 30.

The first image processing unit 8, the communication environment detection unit 11, the execution timing decision unit 22, the speed/distance detection unit 24, and the capsule control unit 72 may also be configured by an integrated circuit such as a processor. Except for the points described above, the configuration shown in FIG. 16 is the same as the configuration shown in FIG. 8.

With a function to temporarily store image data of the periphery of the lesion site, the capsule endoscope 70 can reliably perform imaging of the periphery of the lesion site at which the treatment work is executed at a predetermined position regardless of deterioration in the wireless communication environment. In addition, the capsule endoscope 70 can reliably transfer the image data to the receiving device 30.

The first wireless communication unit 23 receives the work execution condition data from the receiving device 30 in the same manner as in the second embodiment. Accordingly, the capsule endoscope 70 executes the treatment work at a timing based on the work execution condition data when the capsule endoscope 70 moves at a high speed or when the wireless communication environment deteriorates.

In the third embodiment, unlike the second embodiment, an imaging condition of imaging the periphery of the lesion site at which the treatment work is executed is designated by the work execution condition data. The imaging condition includes an imaging position.

Details will be described below using a specific example. In the same manner as in the second embodiment, a positional relationship between the capsule endoscope 70 and the lesion site is shown in FIG. 11. In the following, the capsule endoscope 20 in FIG. 11 is replaced with the capsule endoscope 70.

For example, a distance from the proximity position (P2) to the execution position (P3) of the treatment work is 30 mm, and a distance from the execution position (P3) of the treatment work to the proximity position (P4) is 30 mm. For example, the work execution condition data includes an instruction to capture images from the proximity position (P2) to the proximity position (P4) every time the capsule endoscope 70 moves 1 mm. In this case, 61 images from the proximity position (P2) to the proximity position (P4) are saved in the image data storage unit 71. Specifically, the capsule endoscope 70 starts imaging from the proximity position (P2) at which the work execution condition data is received. The capsule endoscope 70 performs imaging and saving of image data every time a distance represented by distance data from the speed/distance detection unit 24 is updated by 1 mm. The capsule endoscope 70 ends the imaging and the saving of the image data when image data of the 61 images is stored before reaching the proximity position (P4).

The image data stored in the image data storage unit 71 is transmitted to the receiving device 30 at a timing which is different from a timing at which normal image data communication is performed. Specifically, the capsule endoscope 70 performs communication while performing reception confirmation processing (ACK-NACK) ensuring reliable transfer using an idle time of the normal image data communication. A communication method related to the reception confirmation processing is well-known, and thus description thereof will be omitted. In addition, communication methods which are used in the normal image data communication and are not related to the reception confirmation processing are also well-known, and thus description thereof will be omitted.

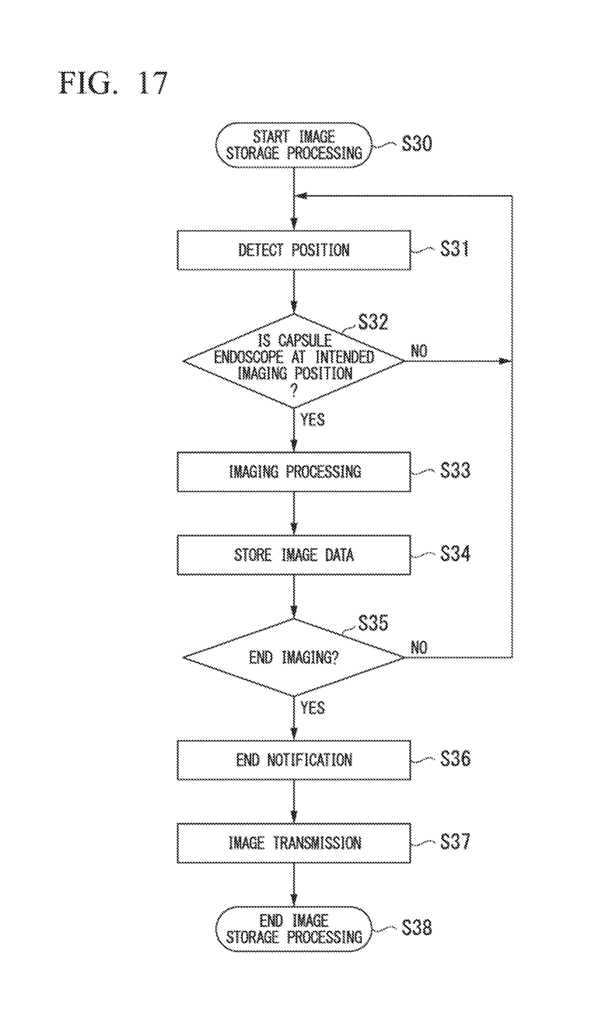

With reference to FIG. 17, image storage processing performed by the capsule endoscope 70 will be described. FIG. 17 shows a procedure of the image storage processing performed by the capsule endoscope 70. The capsule control unit 72 performs the image storage processing by controlling each unit in the capsule endoscope 70.

When the image storage processing starts (S30), the capsule control unit 72 executes position detection (S31). In the position detection (S31), the capsule control unit 72 reads distance data from the speed/distance detection unit 24.

After the distance data is read, the capsule control unit 72 executes determination (S32) of an intended imaging position. In the determination (S32) of an intended imaging position, the capsule control unit 72 determines whether the capsule endoscope 70 moves a predetermined distance from a position at which previous imaging is performed. That is, the capsule control unit 72 determines whether the capsule endoscope 70 is at an intended imaging position. In the present example, the predetermined distance is 1 mm. In the determination (S32) of an intended imaging position after the position detection is performed only once, it is determined that the capsule endoscope 70 is not at the intended imaging position. In addition, after the position detection (S31) is performed twice or more, in the determination (S32) of an intended imaging position until first imaging is performed, it is determined that the capsule endoscope 70 is not at the intended imaging position.

When the capsule endoscope 70 is not at the intended imaging position, the capsule control unit 72 executes the position detection (S31). When the capsule endoscope 70 is at the intended imaging position, the capsule control unit 72 executes imaging processing (S33). In the imaging processing (S33), the capsule control unit 72 selects a predetermined imaging unit and causes the selected imaging unit to perform imaging. Specifically, the imaging unit 21 which performs imaging in the forward direction is selected from the proximity position (P2) to the execution position (P3) of the treatment work. Until the capsule endoscope 70 reaches the proximity position (P4) after the capsule endoscope 70 exceeds the execution position (P3) of the treatment work, the imaging unit 4 which performs imaging in the backward direction is selected. In the imaging processing (S33), the imaging unit 4 or the imaging unit 21 performs imaging at a position in the vicinity of the lesion site and outputs image data of the periphery of the lesion site. In addition, since the imaging processing (S33) is performed in accordance with the determination processing (S32) of an intended imaging position, the imaging unit 4 or the imaging unit 21 performs imaging according to a movement distance.

After the imaging processing (S33) is executed, the capsule control unit 72 executes storage (S34) of the image data. In the storage (S34) of the image data, the capsule control unit 72 causes the image data storage unit 71 to store the image data output from the imaging unit 4 or the imaging unit 21. That is, the image data storage unit 71 temporarily stores the image data output from the imaging unit 4 or the imaging unit 21.

After the image data is stored, the capsule control unit 72 executes imaging end determination (S35). In the imaging end determination (S35), the capsule control unit 72 determines whether an imaging position is the proximity position (P4), thereby determining whether to end imaging. The proximity position (P4) is detected by the method described referring to FIG. 13.

When the imaging position is not the proximity position (P4), imaging continues. In this case, the capsule control unit 72 executes the position detection (S31). When the imaging position is the proximity position (P4), the imaging ends. In this case, the capsule control unit 72 executes end notification (S36). In the end notification (S36), the capsule control unit 72 causes the first wireless communication unit 23 to transmit information representing that the image storage processing ends to the receiving device 30.

After the end notification (S36) is executed, the capsule control unit 72 executes image transmission (S37). In the image transmission (S37), the capsule control unit 72 causes the first wireless communication unit 23 to transmit the image data stored in the image data storage unit 71 to the receiving device 30. That is, the first wireless communication unit 23 transmits the image data stored in the image data storage unit 71 to the receiving device 30. After the image transmission (S37) is executed, the image storage processing ends (S38).

The receiving device 30 notified of an end of the image storage processing through the end notification (S36) starts reception processing of images, which corresponds to the image transmission (S37). The reception processing of images of a periphery of a position at which the treatment work is performed is performed by a communication method related to the reception confirmation processing described above. Details of the communication method are well-known, and thus description thereof will be omitted.

The capsule control unit 72 performs the same determination as the communication environment determination (S13) of FIG. 14, and when deterioration in the wireless communication environment is not detected, the capsule control unit 72 may also perform the image transmission (S37).

In the third embodiment, every time the capsule endoscope 70 moves a predetermined distance, image data of the periphery of the lesion site is temporarily stored. The stored image data is transmitted to the receiving device 30. For this reason, the capsule endoscope 70 can reliably transfer the image data to the receiving device 30 regardless of deterioration in the wireless communication environment.

Embodiments of the present invention have been described above in detail with reference to the drawings. However, a specific configuration is not limited to the embodiments described above, and design changes and the like within a scope not departing from a gist of the present invention are also included.

What is claimed is:

1. A capsule endoscope system, comprising:
a capsule endoscope; and
a receiving device,
wherein the capsule endoscope includes
an imaging unit which performs imaging and outputs image data,
an acceleration sensor which outputs acceleration data, and
a first wireless communication unit which transmits the image data and the acceleration data to the receiving device by wireless communication,
wherein the receiving device includes
a second wireless communication unit which receives the image data and the acceleration data from the capsule endoscope by wireless communication, and
a capsule position detection unit which detects a position of the capsule endoscope based on the image data and the acceleration data,
wherein the first wireless communication unit further receives work execution condition data and work instruction data from the receiving device, the work execution condition data represents a position at which treatment work is performed, and the work instruction data represents an execution instruction of the treatment work,
wherein the capsule endoscope further includes
a speed/distance detection unit which detects a movement speed and a movement distance of the capsule endoscope based on the acceleration data,
an execution timing decision unit which instructs output of an execution command at a timing based on the movement distance and the work execution condition data,
a treatment work unit which performs medication or “collection of tissue or body fluid” based on the execution command, and
a capsule control unit which outputs the execution command to the treatment work unit at a timing at which the work instruction data is received when the movement speed is low, and outputs the execution command to the treatment work unit at a timing at which output of the execution command is instructed by the execution timing decision unit when the movement speed is high,
wherein the receiving device further includes
an operation unit which receives operations of an operator, and
a generation unit which generates the work execution condition data and the work instruction data based on the operations received by the operation unit, and
wherein the second wireless communication unit transmits the work execution condition data and the work instruction data generated by the generation unit to the capsule endoscope.

2. The capsule endoscope system according to claim 1,
wherein the capsule endoscope further includes
an acceleration data storage unit which temporarily stores the acceleration data, and
a communication environment detection unit which detects a wireless communication environment, and
wherein the capsule control unit causes the acceleration data storage unit to store the acceleration data when deterioration in the wireless communication environment is detected by the communication environment detection unit, and causes the first wireless communication unit to transmit the acceleration data stored in the acceleration data storage unit to the receiving device after recovery of the wireless communication environment is detected by the communication environment detection unit.

3. The capsule endoscope system according to claim 2,
wherein the capsule control unit outputs the execution command to the treatment work unit at the timing at which the work instruction data is received when the movement speed is low and deterioration in the wireless communication environment is not detected by the communication environment detection unit, and outputs the execution command to the treatment work unit at the timing at which output of the execution command is instructed by the execution timing decision unit when the movement speed is low and deterioration in the wireless communication environment is detected by the communication environment detection unit.

4. The capsule endoscope system according to claim 2,
wherein the capsule endoscope further includes an image data storage unit which temporarily stores the image data output from the imaging unit,
the imaging unit performs the imaging according to the movement distance at a position based on a position at which the execution command is output, and
the first wireless communication unit further transmits the image data stored in the image data storage unit to the receiving device.

5. A capsule endoscope, comprising:
an imaging unit which performs imaging and outputs image data;
an acceleration sensor which outputs acceleration data;
a first wireless communication unit which transmits the image data and the acceleration data to the receiving device by wireless communication, and receives work execution condition data and work instruction data from the receiving device, the work execution condition data represents a position at which treatment work is performed, and the work instruction data represents an execution instruction of the treatment work;
a speed/distance detection unit which detects a movement speed and a movement distance of the capsule endoscope based on the acceleration data;
an execution timing decision unit which instructs output of an execution command at a timing based on the movement distance and the work execution condition data;
a treatment work unit which performs medication or “collection of tissue or body fluid” based on the execution command; and
a capsule control unit which outputs the execution command to the treatment work unit at a timing at which the work instruction data is received when the movement speed is low, and outputs the execution command to the treatment work unit at a timing at which output of the execution command is instructed by the execution timing decision unit when the movement speed is high.

6. A wireless communication method of a capsule endoscope, comprising:
a step of performing imaging and outputting image data;
a step of outputting acceleration data;
a step of transmitting the image data and the acceleration data to the receiving device by wireless communication, and receiving work execution condition data and work instruction data from the receiving device, the work execution condition data represents a position at which treatment work is performed, and the work instruction data represents an execution instruction of the treatment work;
a step of detecting a movement speed and a movement distance of the capsule endoscope based on the acceleration data;
a step of outputting the execution command to the treatment work unit at a timing at which the work instruction data is received when the movement speed is low, and outputting the execution command to the treatment work unit at a timing at which output of the execution command is instructed by the execution timing decision unit when the movement speed is high; and
a step of performing medication or “collection of tissue or body fluid” based on the execution command.

7. A program for causing a computer of a capsule endoscope to execute steps comprising:
a step of performing imaging and outputting image data;
a step of outputting acceleration data;
a step of transmitting the image data and the acceleration data to the receiving device by wireless communication, and receiving work execution condition data and work instruction data from the receiving device, the work execution condition data represents a position at which treatment work is performed, and the work instruction data represents an execution instruction of the treatment work;
a step of detecting a movement speed and a movement distance of the capsule endoscope based on the acceleration data;
a step of outputting the execution command to the treatment work unit at a timing at which the work instruction data is received when the movement speed is low, and outputting the execution command to the treatment work unit at a timing at which output of the execution command is instructed by the execution timing decision unit when the movement speed is high; and
a step of performing medication or “collection of tissue or body fluid” based on the execution command.
